(19) United States
(12) Patent Application Publication
(10) Pub. No.: US 2022/0125557 A1
(43) Pub. Date: Apr. 28, 2022

(54) DENTAL IMPLANT BODY AND METHOD FOR MANUFACTURING DENTAL IMPLANT BODY

(71) Applicants: Adamant Namiki Precision Jewel Co., Ltd., Tokyo (JP); National Institute of Advanced Industrial Science and Technology, Tokyo (JP)

(72) Inventors: Hikaru Muto, Tokyo (JP); Manabu Fukushima, Aichi (JP); Hideki Hyuga, Aichi (JP); Yuichi Yoshizawa, Aichi (JP)

(21) Appl. No.: 17/647,550

(22) Filed: Jan. 10, 2022

Related U.S. Application Data
(63) Continuation of application No. PCT/JP2020/027661, filed on Jul. 16, 2020.

(30) Foreign Application Priority Data
Jul. 18, 2019 (JP) 2019-132817

(51) Int. Cl. A61C 8/00; C04B 35/486; B28B 11/24; B28B 1/00; C04B 38/00

(52) U.S. Cl. CPC A61C 8/0012; C04B 35/486; B28B 11/243; A61C 8/0093; C04B 2235/606; C04B 38/0045; C04B 38/0054; C04B 2235/3244; C04B 2235/6023; B28B 1/007

(57) ABSTRACT

A dental implant body includes a ceramics sintered body, and the ceramics sintered body is a porous body having blind/continuous holes formed from a surface of the ceramics sintered body and walls formed by the blind/continuous holes. The porosity of the blind/continuous holes may be 50±10%. Further, the diameter of the blind/continuous hole may be equal to or greater than 50 μm and equal to or smaller than 190 μm.

### CROSS-REFERENCE TO RELATED APPLICATION

The present application is a continuation application of International Application No. PCT/JP2020/027661, filed on Jul. 16, 2020, which claims priority to Japanese Patent Application No. 2019-132817, filed on Jul. 18, 2019. The contents of these applications are incorporated herein by reference in their entirety.

### BACKGROUND

### 1. Technical Field

One aspect of the present disclosure relates to a dental implant body and a method for manufacturing the dental implant body.

### 2. Related Art

For a dental implant body (a tooth root portion; hereinafter merely referred to as an “implant body” as necessary) used for dental implant treatment, not only the strength of the implant body alone but also adherence to jawbone cells for joint between the implant body and a bone are required for resistance to biting force and suppression in aging deterioration.

Ceramic has been known as the material of such an implant body. Of ceramic, zirconia (ZrO₂) is specifically suitable for the dental implant body because zirconia has properties such as innoxiousness in terms of health in addition to excellent mechanical properties, chemical durability, and thermal resistance.

For example, one example of such a zirconia dental implant body is described in JP-A-61-146757. JP-A-61-146757 describes an artificial tooth root zirconia implant member made of partially stabilized zirconia containing yttria.

### SUMMARY

A dental implant body includes a ceramics sintered body, and the ceramics sintered body is a porous body having a blind/continuous hole formed from a surface of the ceramics sintered body and a wall formed by the blind/continuous hole.

### BRIEF DESCRIPTION OF DRAWINGS

FIG. 1 shows a SEM observation image of an example of a dental implant body according to one embodiment of the present disclosure, the observation image showing a plane in a direction perpendicular to the direction of formation of blind/continuous holes;

FIG. 2 shows a SEM observation image of the example of the dental implant body according to one embodiment of the present disclosure, the observation image showing a section in the direction of formation of the blind/continuous holes;

FIGS. 3A to 3C show schematic views of the steps of freezing a gel body in the method for manufacturing the dental implant body according to one embodiment of the present disclosure;

FIG. 4 shows a schematic sectional view of a ceramics sintered body forming the dental implant body according to one embodiment of the present disclosure; and

FIG. 5 shows a schematic sectional view of a variation of the ceramics sintered body forming the dental implant body according to one embodiment of the present disclosure.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

If a screw type (screw-shaped) implant body is produced using zirconia as a material, chipping or cracking is caused at a thread, leading to the probability that the strength of joint to a jawbone decreases and the implant body drops from the jawbone.

On the other hand, in the case of using a cylinder type (cylindrical) implant body with no thread, no thread portion is formed, and therefore, there is a probability that the implant body is detached from the jawbone.

One object of the present disclosure is to provide the following dental implant body and the following method for manufacturing the dental implant body. The dental implant body can improve the strength of joint to a jawbone, reduce dropping and detachment from the jawbone, and improve a prognosis.

A dental implant body according to one aspect of the present disclosure includes a ceramics sintered body, and the ceramics sintered body is a porous body having blind/continuous holes formed from a surface of the ceramics sintered body and walls formed by the blind/continuous holes.

A method for manufacturing a dental implant body according to one aspect of the present disclosure includes: producing slurry by dispersing ceramic powder in gelatable liquid; producing a gel body by gelating the slurry; freezing the produced gel body; and drying and sintering the frozen gel body to form, as the dental implant body, a porous body including a ceramics sintered body, having blind/continuous holes formed from a surface of the ceramics sintered body, and having walls formed by the blind/continuous holes.

According to the dental implant body and the dental implant body manufacturing method according to one aspect of the present disclosure, degradation of adherence to jawbone cells can be suppressed, and the strength of joint to the jawbone can be improved. With improvement of the joint strength, dropping and detachment of the dental implant body from the jawbone can be reduced, and the prognosis can be improved.

A dental implant body (the present dental implant body) according to a first feature of the present embodiment includes a ceramics sintered body, and the ceramics sintered body is a porous body having blind/continuous holes formed from a surface of the ceramics sintered body and walls formed by the blind/continuous holes.

In the present dental implant body, a porosity of the blind/continuous holes may be 50±10%, and a diameter of the blind/continuous hole may be equal to or greater than 50 μm and equal to or smaller than 190 μm.

A dental implant body manufacturing method (the present manufacturing method) according to a second feature of the present embodiment includes: producing slurry by dispersing ceramic powder in gelatable liquid; producing a gel body by gelating the slurry; freezing the produced gel body; and drying and sintering the frozen gel body to form, as the dental implant body, a porous body including a ceramics sintered body, having blind/continuous holes formed from a surface of the ceramics sintered body, and having walls formed by the blind/continuous holes.

The present manufacturing method may further include: setting a concentration of the ceramic powder dispersed in the gelatable liquid to equal to or higher than 5% and equal to or lower than 65%; setting a temperature when the gel body is frozen to a range of equal to or higher than −40° C. and equal to or lower than −10° C.; setting a porosity of the blind/continuous holes in the porous body to 50±10%; and setting a diameter of the blind/continuous hole to equal to or greater than 50 μm and equal to or smaller than 190 μm.

According to these configuration and manufacturing method, degradation of adherence to the jawbone cells can be suppressed or prevented, and the strength of joint to the jawbone can be improved. With improvement of the joint strength, dropping and detachment of the dental implant body from the jawbone can be reduced or prevented, and the prognosis (a progress after dental implant treatment) can be also improved.

Note that in the present embodiment, the blind/continuous hole indicates a pore formed to appear at a surface of a dental implant body. Further, the continuous hole indicates a pore penetrating a dental implant body to a back surface as another surface facing a front surface. On the other hand, the blind hole indicates a pore not penetrating a dental implant body to a back surface.

In the present dental implant body, the ceramics sintered body may be made of zirconia.

In the present manufacturing method, the ceramics sintered body may be made of zirconia.

According to these configuration and manufacturing method, the ceramics sintered body is made of zirconia. Thus, the ceramics sintered body is suitable for the dental implant body because such a ceramics sintered body has properties such as chemical durability, thermal resistance, and innoxiousness in terms of health. Further, favorable mechanical properties (mechanical strength and processability) are obtained.

In the present dental implant body, the blind/continuous holes may be formed in a certain direction and the walls may be densely formed.

In the present manufacturing method, the freezing the gel body may include growing ice crystals in a certain direction in the gel body to form the blind/continuous holes in the certain direction and densely forming the walls.

According to these configuration and manufacturing method, the direction of formation of the blind/continuous holes is one direction, and therefore, the walls can be more densely formed. Thus, the dental implant body can be obtained, which has high mechanical properties suitable for dental use while having the blind/continuous holes.

Note that in the present embodiment, the dense wall includes not only a wall with no holes, micropores, or nanopores, but also a wall with a density of equal to or higher than 99%, for example.

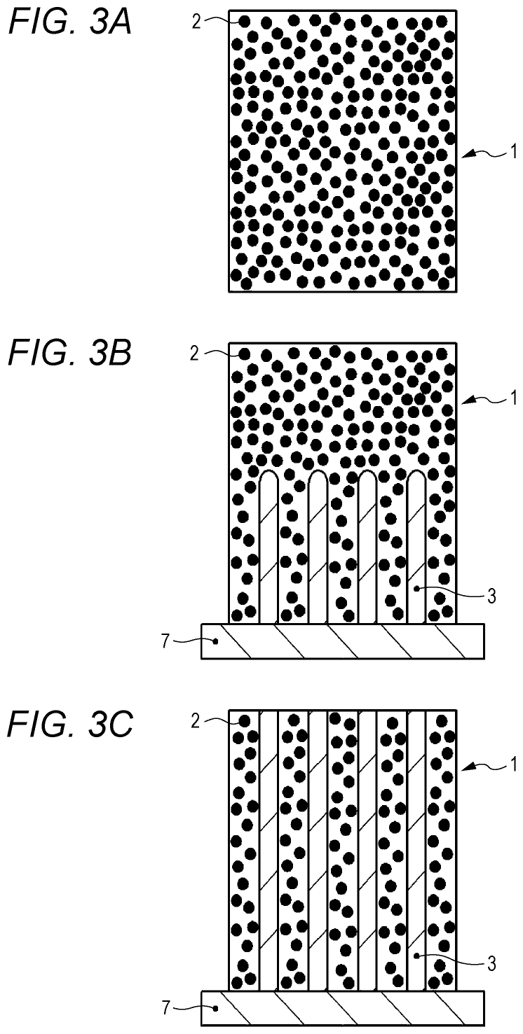

Hereinafter, the method for manufacturing a dental implant body according to the present embodiment will be described in detail with reference to FIGS. 3A to 5, and the dental implant body obtained by such a manufacturing method will be described as needed. In the manufacturing method according to the present embodiment, gelatable polymeric liquid is first prepared. Slurry is produced in such a manner that ceramic powder is dispersed in the liquid at a powder concentration of equal to or higher than 5% and equal to or lower than 65%.

The ceramic powder dispersed in the liquid contains zirconia (ZrO₂), and for example, is substantially made of zirconia. Further, the ceramic powder is granulated. The average particle size of the ceramic powder is set to a range of 0.01 μm (10 nm) to 0.08 μm (80 nm). If the average particle size is smaller than 0.01 μm, it is not preferred because the ceramic powder is difficult to be handled and workability is degraded. On the other hand, if the average particle size exceeds 0.08 μm, it is not preferred because the ceramic powder is easily precipitated in the slurry and it is difficult to stably obtain the slurry.

The water content of the slurry is 35 wt % to 95 wt %. If the water content is lower than 35 wt %, the ceramic powder is aggregated and easily precipitated, and accordingly, a stable dispersion state is difficult to be held. On the other hand, if the water content exceeds 95 wt %, the density of a ceramic molded body after water has been sublimed into ice crystals is extremely low and it is difficult for the ceramic molded body to satisfy a strength as the dental implant body.

Next, a gel body is produced in such a manner that the produced slurry is gelated. Gelation indicates that the slurry in which the ceramic powder is dispersed is solidified. The gel body is formed with a cylindrical outer shape. FIG. 3A schematically shows a gel body 1. Black circles in the gel body 1 indicate dispersed ceramic powder 2.

Next, the produced gel body 1 is frozen at a range of equal to or higher than −40° C. and equal to or lower than −10° C. In freezing of the gel body 1, a bottom surface of the gel body 1 contacts a copper or aluminum freezing plate 7 as shown in FIG. 3B. Then, by heat transfer by means of the freezing plate 7, the gel body 1 is cooled in a certain direction (an upward direction in FIG. 3B) from the bottom surface to the other side. Note that FIG. 3B is a sectional view of the gel body 1 cut along an optional section. In this figure, the section of the gel body 1 in which the ceramic powder 2 is dispersed is shown without hatching for the sake of viewability.

Since the gel body 1 is frozen in the certain direction from the bottom surface contacting the freezing plate 7, multiple ice crystals 3 frozen in a frost column shape from water without the ceramic powder 2 being dispersed are formed in the certain direction in the gel body 1 as shown from FIG. 3B to FIG. 3C. Note that FIG. 3C is also a sectional view of the gel body 1 cut along an optional section as in FIG. 3B. In this figure, the section of the gel body 1 in which the ceramic powder 2 is dispersed is shown without hatching for the sake of viewability. In FIGS. 3B and 3C, hatched portions indicate the ice crystals 3.

Meanwhile, the ceramic powder 2 is unevenly distributed in regions of the gel body 1 other than the ice crystals 3, as shown in FIGS. 3B and 3C. Water expands due to freezing. For this reason, the regions in which the ceramic powder is unevenly distributed are pushed and compressed by the ice crystals 3. Due to such compression, the regions in which the ceramic powder is unevenly distributed are densified, and later-described walls are formed accordingly. Note that the thickness (the dimension in the upper-lower direction in FIGS. 3A to 3C) of the gel body 1 shown in FIG. 3A to 3C is set to 1.2 μm to 130 μm.

As described above, freezing of the gel body 1 includes growing the ice crystals 3 in the certain direction in the gel body 1 to form blind/continuous holes in the certain direction and densely forming the walls.

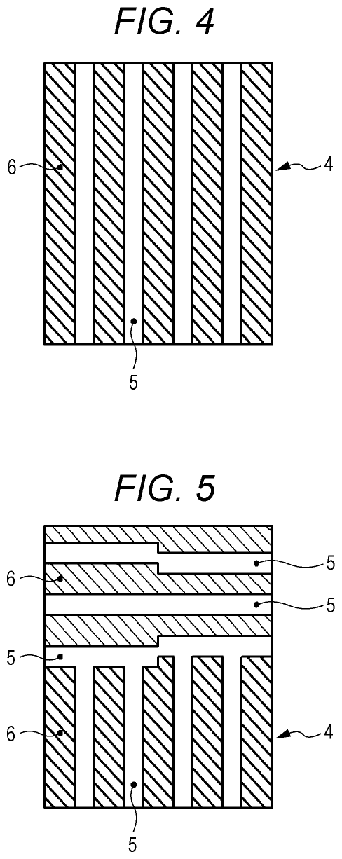

Next, the frozen gel body is dried in atmosphere, and accordingly, the ice crystals 3 are sublimed to obtain a ceramic molded body. Thereafter, the ceramic molded body is sintered to form a ceramics sintered body 4 shown in FIG. 4. Note that FIG. 4 is a sectional view of the ceramics sintered body 4 cut along an optional section. A sintering method is atmospheric sintering, a heating temperature is 2° C./min to 10° C./min, a sintering temperature is 1300° C. to 1500° C., atmosphere is atmospheric air, a pressure is an ordinary pressure, and a sintering time is one hour to four hours.

As described above, the ice crystals 3 are sublimed and sintered thereafter. Accordingly, a porous body including the ceramics sintered body 4 can be formed as shown in FIG. 4. This porous body has blind/continuous holes 5 formed from a surface of the ceramics sintered body 4 and a wall 6 formed between adjacent ones of the blind/continuous holes 5. In the ceramics sintered body 4 shown as an example in FIG. 4, the continuous holes 5 are formed as pores, and the wall 6 is formed between adjacent ones of the continuous holes 5.

The blind/continuous hole indicates a pore formed to appear at a surface of a dental implant body including a ceramics sintered body. Further, the continuous hole indicates, as shown in FIG. 4, a pore penetrating a dental implant body to a back surface as another surface facing a front surface. On the other hand, the blind hole indicates a pore not penetrating a dental implant body to a back surface.

Since the blind/continuous holes are formed from the surface, jawbone cell tissues can smoothly enter the blind/continuous holes when the dental implant body formed using the porous body as described above is fixed to a jawbone. Thus, degradation of adhesion of the dental implant body to jawbone cells can be suppressed without the need for forming a thread at the dental implant body, and the strength of j oint to the jawbone can be improved. With improvement of the joint strength, dropping and detachment of the dental implant body from the jawbone can be reduced. Further, a prognosis (a progress after dental implant treatment) can be also improved. In addition, there are no concerns about chipping and cracking due to thread formation.

Further, in the present embodiment, the porosity of the blind/continuous holes is set to 50±10%, and the diameter of the blind/continuous hole is set to equal to or greater than 50 μm and equal to or smaller than 190 μm. That is, in the present embodiment, all of the blind/continuous holes formed from the surface of the ceramics sintered body are formed such that the diameters thereof fall within a range of equal to or greater than 50 μm and equal to or smaller than 190 μm. As described above, the numerical values of the porosity and the diameter are set and the single porous body achieves these numerical values all at once, and therefore, the jawbone cell tissues can enter all of the blind/continuous holes. Thus, degradation of adhesion of the dental implant body to the jawbone cells can be suppressed without the need for forming the thread at the dental implant body, and the strength of joint to the jawbone can be further improved. With improvement of the joint strength, dropping and detachment of the dental implant body from the jawbone can be reduced, and the prognosis can be also improved.

The porosity is 50% as a center value, and changes within a range of ±10%. It has found that if the porosity exceeds 50%+10% (i.e., 60%), the dental implant body formed using the porous body is less likely to satisfy a strength for dental use for which resistance to biting force in daily diet and suppression in aging deterioration due to biting are required. On the other hand, it has found that if the porosity is less than 50%-10% (i.e., 40%), the number ofjawbone cell tissues which can enter the blind/continuous holes decreases in association with a decrease in the porosity and adhesion of the dental implant body to the jawbone cells is degraded.

Further, it has found that if the diameter of the blind/continuous hole exceeds 190 μm, the diameter is too expanded, the growth rate of the jawbone cells in the blind/continuous hole decreases, and the strength ofjoint between the dental implant body and the jawbone decreases. On the other hand, it has found that if the diameter of the blind/continuous hole is smaller than 50 μm, the jawbone cell tissues are difficult to enter the blind/continuous holes, adhesion of the dental implant body to the jawbone cells is degraded, and the strength of joint between the dental implant body and the jawbone decreases.

As described above, it has confirmed that in the dental implant body formed using the porous body, the porosity of the blind/continuous holes is preferably set to 50±10% and the diameter of the blind/continuous hole is preferably set to equal to or greater than 50 μm and equal to or smaller than 190 μm for suppressing degradation of adhesion of the dental implant body to the jawbone cells and reducing dropping and detachment of the dental implant body from the jawbone.

The porosity can be measured by a mercury intrusion technique using a mercury porosimeter. Moreover, the diameter of the blind/continuous hole is measured using a scanning electron microscope (SEM) observation image.

In the present embodiment, it has found that as the conditions for achieving a blind/continuous hole porosity of 50±10% and a blind/continuous hole diameter of equal to or greater than 50 μm and equal to or smaller than 190 μm all at once, the concentration of the ceramic powder dispersed in the gelatable liquid is set to equal to or higher than 5% and equal to or lower than 65% and a gel body freezing temperature is set to a range of equal to or higher than −40° C. and equal to or lower than −10° C. If either one of the concentration range of the ceramic powder 2 or the freezing temperature of the gel body 1 falls outside the above-described range, both of the porosity and the diameter are difficult to be set within desired ranges.

Further, if the concentration of the ceramic powder 2 is lower than 5%, the ceramic molded body is less likely to satisfy the strength as the dental implant body due to an extremely-low density of the ceramic molded body. If the concentration of the ceramic powder 2 exceeds 65%, the ceramic powder is aggregated and easily precipitated, and the stable dispersion state is difficult to be held.

If the gel body freezing temperature reaches lower than −40° C., the entire gel body is frozen before the ice crystals are formed in the frost column shape. Thus, the ceramic powder is difficult to be unevenly distributed in the gel body regions other than the ice crystals. In addition, if the gel body freezing temperature exceeds −10° C., the ice crystals are difficult to be formed in the gel body.

Further, in the present embodiment, according to the porous body used for the dental implant body and the method for manufacturing such a porous body, the walls 6 are more preferably densely formed in such a manner that the gel body 1 is frozen such that the ice crystals 3 are grown in the certain direction in the gel body 1 as shown in FIGS. 3A to 3C and the blind/continuous holes 5 are accordingly formed in the certain direction as shown in FIG. 4. The dense wall includes not only a wall with no holes, micropores, or nanopores, but also a wall with a density of equal to or higher than 99%. A reason why the dense wall is not limited to one with a density of 100% is that in addition to the frost-columnar ice crystals, ice having an extremely-smaller diameter than that of the ice crystal is formed in the walls in the gel body at the above-described freezing step in some cases. Such ice is also sublimed upon drying of the gel body, and therefore, fine holes or pores are formed after sublimation of the ice and holes, micropores, or nanopores are formed in the walls in some cases.

Note that the blind/continuous holes formed in the certain direction indicate those formed only in substantially one direction from the front surface to the back surface (from up to down in FIG. 4) of the ceramics sintered body 4, such as the continuous holes 5 of FIG. 4.

For formation of the blind/continuous holes in the certain direction, the ice crystals are formed in the certain direction in the gel body. As a result of study conducted by the applicant of the present application, the conditions for formation of the ice crystals in the certain direction are that the gel body is, by heat transfer, gradually frozen from one location to the other location along the certain direction at a range of equal to or higher than −40° C. and equal to or lower than −10° C.

In FIGS. 3B and 3C, the surface of the gel body 1 contacting the freezing plate 7 is one location from which freezing is started. From such a surface, the gel body 1 is, by heat transfer, gradually frozen to the upper surface as the other location along the certain direction (the upward direction in FIGS. 3B and 3C).

The direction of formation of the blind/continuous holes is one direction so that the ceramic powder can be unevenly distributed with regularity and the ice crystals can uniformly apply compression force to the entire surfaces of the walls. Thus, the walls can be more densely formed. Thus, the dental implant body can be obtained, which has high mechanical properties (mechanical strength and processability) suitable for dental use while having the blind/continuous holes. Consequently, in addition to setting of the porosity and diameter of the blind/continuous holes, the direction of formation of the blind/continuous holes is, for the dental implant body and the method for manufacturing the dental implant body, more preferably set to one direction.

The blind/continuous holes may be formed in the right-left direction, an oblique direction, or a combination thereof in the ceramics sintered body. In a variation example shown in FIG. 5, multiple continuous holes 5 are formed from side surfaces in the horizontal direction as viewed in the figure at the upper half of the ceramics sintered body 4. Further, multiple continuous holes 5 are formed in the vertical direction as viewed in the figure at the lower half of the ceramics sintered body 4, as in FIG. 4. Note that FIG. 5 is a sectional view of the ceramics sintered body 4 cut along an optional section. Of the multiple continuous holes 5 formed in the horizontal direction, the lowermost continuous hole 5 communicates with the continuous holes 5 formed in the vertical direction.

In manufacturing of the ceramics sintered body 4 of FIG. 5, the freezing plate 7 is arranged on the bottom surface of the gel body to form the continuous holes 5 in the vertical direction. Further, a freezing agent is arranged on the entire circumference of the side surfaces of the upper half of the gel body to form the multiple continuous holes 5 from the side surfaces to the center of the gel body. Thus, in some cases, the continuous holes 5 are formed such that the positions of openings formed at both side surfaces are shifted from each other in the upper-lower direction, as in the uppermost and lowermost ones of the multiple continuous holes 5 in the horizontal direction as shown in FIG. 5. In other cases, the continuous holes 5 are formed such that the positions of the openings formed at both side surfaces are coincident with each other in the upper-lower direction, as in the continuous hole 5 formed in the middle.

Further, in the present embodiment, zirconia is used as the ceramic powder which is a raw material. Thus, the ceramics sintered body is made of zirconia. Since the ceramics sintered body is made of zirconia, the ceramics sintered body is suitable for the dental implant body because such a ceramics sintered body has properties such as chemical durability, thermal resistance, and innoxiousness in terms of health. Further, favorable mechanical properties (mechanical strength and processability) are obtained.

Considering application of zirconia to the dental implant body as a biological reinforcement member, yttria (yttrium oxide: Y₂O₃) containing zirconia is more preferred for ensuring higher mechanical strength. Specific examples of yttria containing zirconia include 2Y zirconia (2 mol % yttria containing zirconia), 2.5Y zirconia (2.5 mol % yttria containing zirconia), 3Y zirconia (3 mol % yttria containing zirconia), and 8Y zirconia (8 mol % yttria containing zirconia).

The color of the implant body obtained as described above is white, and the Vickers hardness of the implant body is 10 GPa in one direction in which the blind/continuous holes are formed and is 3.5 GPa in other directions. The implant body can be formed as a one-piece type or a two-piece type with another abutment (support).

Hereinafter, an example according to the present embodiment will be described. Note that the present embodiment is not limited only to the following example.

### EXAMPLE

Hereinafter, the method for manufacturing a dental implant body according to the present example will be described. First, gelatable polymeric liquid was prepared. Slurry was produced in such a manner that granulated zirconia powder is dispersed in the liquid at a powder concentration of 35%. In the present example, polycrystalline zirconia as a dense body containing no yttria and having a density of 99% was used. Such polycrystalline zirconia was obtained by sintering for two hours at a temperature range of 1350° C. to 1450° C.

Next, a gel body was produced in such a manner that the produced slurry is gelated. Further, a copper freezing plate 7 contacted a bottom surface of the gel body as in FIG. 3B. Then, the gel body was frozen at −20° C. in such a manner that the gel body is, by heat transfer by means of the freezing plate 7, cooled in a certain direction from the bottom surface to the other side. Accordingly, multiple ice crystals formed in the certain direction as in FIG. 3C were obtained.

Next, the frozen gel body was dried in atmosphere, and accordingly, the ice crystals were sublimed to obtain a ceramic molded body. Further, the ceramic molded body was sintered to form a ceramics sintered body. A sintering method was atmospheric sintering, a heating temperature was 2° C./min, a sintering temperature was 1350° C., atmosphere was atmospheric air, a pressure was an ordinary pressure, and a sintering time was two hours.

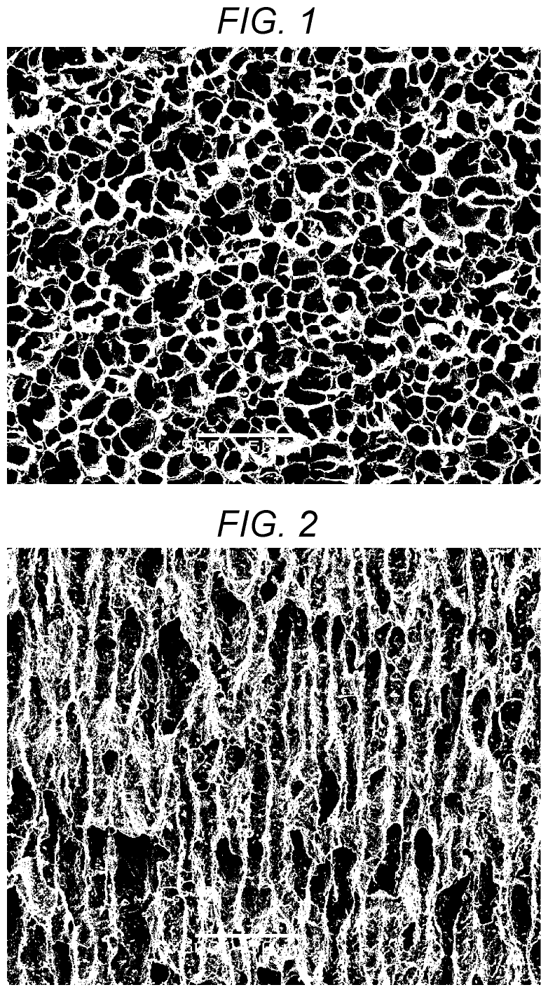

SEM observation images of the obtained ceramics sintered body are shown in FIGS. 1 and 2. FIG. 1 shows the observation image in a plane perpendicular to the direction of formation of the blind/continuous holes. FIG. 2 shows the observation image in a section in the direction of formation of the blind/continuous holes. From FIG. 1, it has confirmed that the blind/continuous holes are formed from a surface of the ceramics sintered body. From FIG. 2, it has also confirmed that the blind/continuous holes are formed in the certain direction (the upper-lower direction in FIG. 2). Moreover, it has also confirmed that a wall density is 99% and walls are densely formed.

The porosity of the blind/continuous holes of the ceramics sintered body was measured by a mercury intrusion technique using a mercury porosimeter. As a result, it has found that the porosity is 60%. Moreover, the diameter of the blind/continuous hole was observed using the SEM observation images. As a result, it has also found that all diameters observed at randomly selected multiple locations of the SEM observation images are within a range of equal to or greater than 50 μm and equal to or smaller than 190 μm.

Jawbone cell tissues of multiple subjects randomly selected regardless of sex and age adhered to a surface of the ceramics sintered body. As a result, entrance of the jawbone cell tissues into the blind/continuous holes was observed. Further, it has confirmed that the jawbone cell tissues having entered the blind/continuous holes are held adhered to the surface of the ceramics sintered body without detachment.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A dental implant body comprising:
a ceramics sintered body,
wherein the ceramics sintered body is a porous body having a blind/continuous hole formed from a surface of the ceramics sintered body and a wall formed by the blind/continuous hole.

2. The dental implant body according to claim 1, wherein
a porosity of the blind/continuous hole is 50±10%, and
a diameter of the blind/continuous hole is equal to or greater than 50 μm and equal to or smaller than 190 μm.

3. The dental implant body according to claim 1, wherein
the ceramics sintered body is made of zirconia.

4. The dental implant body according to claim 2, wherein
the ceramics sintered body is made of zirconia.

5. The dental implant body according to claim 1, wherein
the blind/continuous hole is formed in a certain direction, and the wall is densely formed.

6. The dental implant body according to claim 2, wherein
the blind/continuous hole is formed in a certain direction, and the wall is densely formed.

7. The dental implant body according to claim 3, wherein
the blind/continuous hole is formed in a certain direction, and the wall is densely formed.

8. The dental implant body according to claim 4, wherein
the blind/continuous hole is formed in a certain direction, and the wall is densely formed.

9. A method for manufacturing a dental implant body, comprising:
producing slurry by dispersing ceramic powder in gelatable liquid;
producing a gel body by gelating the slurry;
freezing the produced gel body; and
drying and sintering the frozen gel body to form, as the dental implant body, a porous body including a ceramics sintered body, having a blind/continuous hole formed from a surface of the ceramics sintered body, and having a wall formed by the blind/continuous hole.

10. The method for manufacturing the dental implant body according to claim 9, further comprising:
setting a concentration of the ceramic powder dispersed in the gelatable liquid to equal to or higher than 5% and equal to or lower than 65%;
setting a temperature when the gel body is frozen to a range of equal to or higher than −40° C. and equal to or lower than −10° C.;
setting a porosity of the blind/continuous hole in the porous body to 50±10%; and
setting a diameter of the blind/continuous hole to equal to or greater than 50 μm and equal to or smaller than 190 μm.

11. The method for manufacturing the dental implant body according to claim 9, wherein
the ceramics sintered body is made of zirconia.

12. The method for manufacturing the dental implant body according to claim 10, wherein
the ceramics sintered body is made of zirconia.

13. The method for manufacturing the dental implant body according to claim 9, wherein
the freezing the gel body includes growing an ice crystal in a certain direction in the gel body to form the blind/continuous hole in the certain direction and densely forming the wall.

14. The method for manufacturing the dental implant body according to claim 10, wherein
the freezing the gel body includes growing an ice crystal in a certain direction in the gel body to form the blind/continuous hole in the certain direction and densely forming the wall.

15. The method for manufacturing the dental implant body according to claim 11, wherein
the freezing the gel body includes growing an ice crystal in a certain direction in the gel body to form the blind/continuous hole in the certain direction and densely forming the wall.

16. The method for manufacturing the dental implant body according to claim 12, wherein
the freezing the gel body includes growing an ice crystal in a certain direction in the gel body to form the blind/continuous hole in the certain direction and densely forming the wall.
